(19) United States
(12) Patent Application Publication
(10) Pub. No.: US 2015/0018582 A1
(43) Pub. Date: Jan. 15, 2015

(54) DIRECT DIMETHYL ETHER SYNTHESIS FROM SYNTHESIS GAS

(71) Applicant: LINDE AKTIENGESELLSCHAFT, MUNCHEN (DE)

(72) Inventors: Nicole Schodel, Munchen (DE); Ernst Haidegger, Riemerling (DE); Holger Schmigalle, Geretsried (DE); Christian Thaller, Munchen (DE); Harald Schmaderer, Wolfratshausen (DE); Akos Tota, Munchen (DE)

(73) Assignee: LINDE AKTIENGESELLSCHAFT, MUNCHEN (DE)

(21) Appl. No.: 14/375,479

(22) PCT Filed: Jan. 15, 2013
(86) PCT No.: PCT/EP2013/000103

§ 371 Date: Jul. 30, 2014

(30) Foreign Application Priority Data
Jan. 31, 2012 (DE) 102012001804.5

(51) Int. Cl. C07C 41/01

(52) U.S. Cl. CPC C07C 41/01
USPC 568/671

(57) ABSTRACT

The invention concerns processes for preparing dimethyl ether from synthesis gas, comprising a synthesis step, wherein synthesis gas comprising hydrogen and carbon monoxide is converted into dimethyl ether and carbon dioxide, and a separation step, wherein unconverted synthesis gas is separated from carbon dioxide, wherein unconverted synthesis gas is separated from carbon dioxide by a membrane.

The invention relates to a process for preparation of dimethyl ether from synthesis gas.

A process of this type comprises a synthesis step, wherein synthesis gas comprising hydrogen and carbon monoxide is converted into dimethyl ether and carbon dioxide, and a separation step, wherein unconverted synthesis gas is separated from carbon dioxide.

A direct synthesis of dimethyl ether (DME) involves a main reaction wherein an H₂/CO mixture having a stoichiometrically preferred ratio of 1:1 between H₂ and CO is converted into DME and CO₂ over a bifunctional catalyst. In addition to the main reaction there is also a reaction that by-produces methanol and water at low selectivity. Since the reactions are equilibrium reactions, the H₂CO mixture is never fully converted. The reactor effluent stream thus consists essentially of the components DME, CO₂, CO, H₂, H₂O and methanol. To maximize process economics, it is desirable that it should be possible for the gas components CO, H₂ and CO₂ to be separated and further used as materials.

The separation of CO₂ from unconverted synthesis gas is the key problem in the separation part of a single-stage DME plant. Prior art solution attempts have disadvantages.

The DME synthesis reaction can be operated with excess hydrogen in order that the formation of carbon dioxide may be suppressed. After the synthesis reaction, hydrogen, CO and CO₂ are separated from the other products—DME, H₂O and methanol—and fed back into the synthesis reaction. However, operating the DME reaction with excess hydrogen leads to a distinct loss of productivity.

Alternatively, the DME synthesis can be run close to the stoichiometric optimum, but then the carbon dioxide has to be recycled into the synthesis gas generation process (based on methane or natural gas) and the carbon monoxide and hydrogen have to be recycled into the DME synthesis reaction. Separate CO₂ removal from the H₂/CO mixture is so costly and inconvenient in terms of apparatus and process-control requirements as to undo the advantages of the direct DME synthesis over an indirect DME synthesis.

Absorptive separation of CO₂, for example, is always faced with the following two problems: on the one hand, the absorbent has to have good CO₂ solubility under moderate conditions while, on the other hand, it should have a very low vapour pressure in order that it may be regenerated at low cost and inconvenience. Mostly it is methanol which is used in commercial processes for removing CO₂ from gas streams, but the process is operated at about −30° C., which incurs appreciable costs due to the provision of cold. The solubility of CO₂ in DME is distinctly higher than in methanol. However, DME has a high vapour pressure under moderate conditions. As a result, a certain DME fraction is recycled with the synthesis gas into the DME synthesis reaction, reducing the productivity. In the course of further processing, the high vapour pressure requires a compromise to be made between elevated pressure and low top-of-condenser temperature in order that the CO₂-DME mixture may be separated by distillation.

U.S. Pat. No. 6,458,856 describes using a DME/methanol mixture as a scrubbing solvent. However, the problems described persist beyond the use of this scrubbing solvent mixture.

Against this background, the problem addressed by the present invention is therefore that of providing a technically simple and economically feasible process for synthesizing dimethyl ether from synthesis gas.

This problem is solved by a process having the features of claim 1.

In said process, the unconverted synthesis gas is separated from carbon dioxide by a membrane.

A membrane for the purposes of the invention is a separation layer whereby two (possibly homogeneous) phases of differing composition are separated from each other. The membrane may be semi-permeable or selectively permeable for certain materials. The membrane may consist of glass or manufactured materials such as ceramics or one or more polymers. Useful polymers include, for example, multiblock copolymers of polyethylene glycol and polyamide, or silicone-based polymers such as polydimethylsiloxane for example. The membrane may be configured as a flat membrane in the form of a foil or as a hollow (fibre) membrane in the form of a hose or tube.

In one embodiment of the invention, the membrane is characterized by a high separation factor for carbon dioxide or synthesis gas. Separation factor for the purposes of the invention is the ratio of the concentration of a material in the permeate and the feed concentration of the material into the membrane separation. A preferred separation factor for carbon dioxide or synthesis gas is 0.7, more preferably 0.8 or 0.9.

A particularly preferred membrane has different separation factors for carbon dioxide and synthesis gas. This membrane has different permeabilities for carbon dioxide and synthesis gas and/or different permeation rates for carbon dioxide and synthesis gas. A membrane of this type is configured to enrich carbon dioxide in the permeate and synthesis gas in the retentate and/or enrich carbon dioxide in the retentate and synthesis gas in the permeate. Enriching for the purposes of the invention is to be understood as meaning in particular that the membrane separation has the effect that at least 60%, 70%, 80% or 90% of the feed materials end up in the permeate or in the retentate.

In a preferred embodiment of the invention, the membrane is characterized by a high gas flux for carbon dioxide, especially by a high permeability for carbon dioxide. A high gas flux for the purposes of the invention is a high permeation rate for carbon dioxide.

In a further preferred embodiment of the invention, the membrane is characterized by high gas flux for dimethyl ether, especially by a high permeability for dimethyl ether. A high gas flux for the purposes of the invention is a high permeation rate for dimethyl ether.

In a further preferred embodiment, the membrane consists of at least one polymer, especially of polydimethylsiloxane. A polymer for the purposes of the invention may be more particularly a homopolymer or a copolymer. The membrane may also consist of two or more different polymers, so-called polymer alloys.

In a further embodiment of the invention, the separation step of carbon dioxide and unconverted synthesis gas produces a predominantly hydrogen- and carbon monoxide-containing retentate and a predominantly dimethyl ether- and carbon dioxide-containing permeate.

A predominantly hydrogen- and carbon monoxide-containing retentate for the purposes of the invention is to be understood as meaning more particularly that the retentate consists of hydrogen and carbon monoxide to an extent of not less than 60%, 70% or 80% (volume percent). Similarly, a predominantly dimethyl ether- and carbon dioxide-containing permeate is to be understood as meaning more particularly that the permeate consists of carbon dioxide and dimethyl ether to an extent of not less than 60%, 70% or 80% (volume percent).

In a further embodiment of the invention, the predominantly hydrogen- and carbon dioxide-containing retentate is fed into the synthesis step. Feeding the synthesis gas back into the synthesis step leads to an increased yield for the reaction and a reduction in the amount of waste product generated.

In a further embodiment, carbon dioxide is separated from the predominantly dimethyl ether- and carbon dioxide-containing permeate. Carbon dioxide can be separated from the permeate by conventional separation processes such as distillation for instance. Alternatively, carbon dioxide can also be separated from the permeate by amine or alkali metal carbonate scrubs, scrubs with organic solvents such as methanol, N-methyl-2-pyrrolidone or polyethylene glycol dimethyl ether or by using a membrane.

In a further embodiment, carbon dioxide separated from the permeate is used for production of synthesis gas, wherein carbon dioxide and methane are converted into hydrogen and carbon monoxide.

The separation step wherein unconverted synthesis gas is separated from carbon dioxide is advantageously preceded by an optional separation being effected between the liquid and gaseous phases.

Illustrative embodiments of the invention will now be more particularly described with reference to the figures and the related description to elucidate further details and advantages of the invention.

Furthermore dimethyl ether can be converted into a product containing olefins, particularly ethylene and/or propylene, wherein dimethyl ether (i.e. the dimethyl ether-containing permeate) is fed to the synthesis of olefins directly or only carbon dioxide is separated from the permeate, before the permeate is fed to the synthesis of olefins.

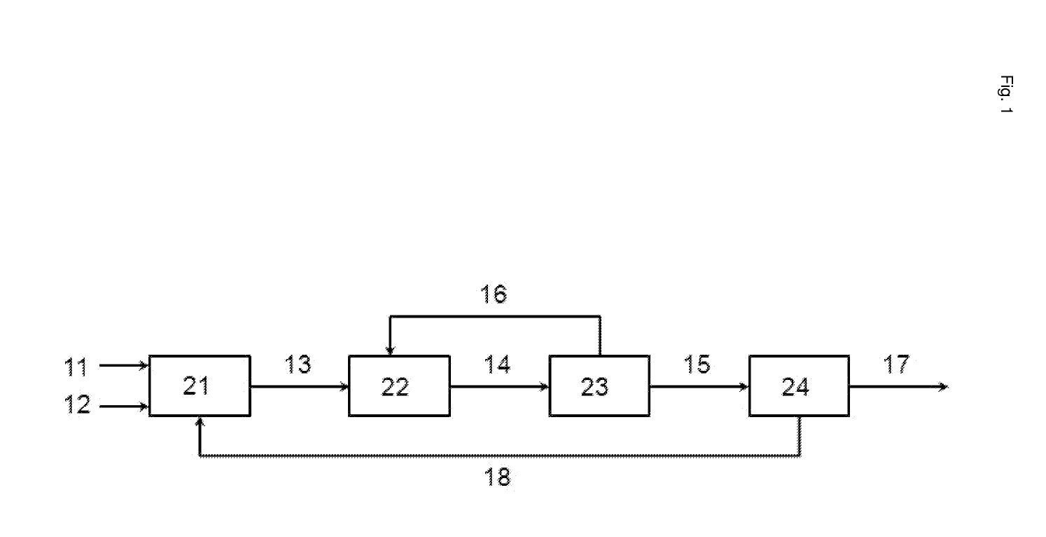

FIG. 1 shows a diagram of the process according to the invention.

### EXAMPLE 1

The present invention is more particularly concerned with the separation part of a process for direct DME synthesis (FIG. 1).

The direct dimethyl ether process 22 converts synthesis gas 13 into dimethyl ether and carbon dioxide. The synthesis product gas stream 14 contains not just the products but also unconverted synthesis gas. This synthesis gas is separated with a polymer membrane from dimethyl ether and carbon dioxide at 23, while the synthesis gas-containing retentate 16 is returned back into the DME synthesis 22. Carbon dioxide 18 is subsequently separated from the remaining dimethyl ether-carbon dioxide-containing permeate 15 and used for synthesis gas production 21 wherein methane 11 and carbon dioxide 12, 18 is converted into synthesis gas 13.

The separation 23 of gas streams by means of polymer membranes is an alternative to the conventional separation processes, such as distillation, adsorption or absorption. It is particularly important for the economics of this separation process that the membrane used has a high separation factor and a high gas flux.

High-permeability polymer membranes, such as PDMS membranes (polydimethylsiloxane) are very useful to split the composition of matter 14 from the DME reactor 22 (H2CO/CO2/DME/methanol) into the following two fractions:
retentate 16: H2, CO, +traces DME and CO2
permeate 15: DME, CO2, methanol +traces H₂ and CO.
   The separation task is significantly facilitated as a result. The retentate fraction 16 can thus be fed directly into the reaction part 22. The permeate fraction 15 is distillatively separated via conventional separation processes 24 and CO₂ 18 is returned into the synthesis gas part 21. The purification requirements of DME 17 are substantially dependent on the intended use (propellant, solvent, LPG admixture, fuel, feedstock for olefin synthesis).

List of reference signs:

| | |
|---|---|
| 11 | Methane |
| 12 | CO₂ |
| 13 | Synthesis gas (H₂, CO) |
| 14 | Synthesis product gas stream (DME, CO₂, H₂, CO, MeOH) |
| 15 | Permeate (DME, CO₂) |
| 16 | Retentate (H₂, CO) |
| 17 | DME |
| 18 | CO₂ |
| 21 | Synthesis gas production |
| 22 | Direct DME synthesis |
| 23 | Separation of CO₂ and synthesis gas |
| 24 | Separation of CO₂ and DME |

1. Process for preparation of dimethyl ether (17) from synthesis gas (13), comprising
a synthesis step (22), wherein synthesis gas (13) comprising hydrogen and carbon monoxide is converted into dimethyl ether and carbon dioxide, and
a separation step (23), wherein unconverted synthesis gas is separated from carbon dioxide,
characterized in that,
unconverted synthesis gas is separated from carbon dioxide by a membrane.

2. Process according to claim 1, characterized in that the membrane has a separation factor for carbon dioxide or synthesis gas, which is at least 0.7, preferably 0.8, more preferably 0.9.

3. Process according to claim 1, characterized in that the membrane has a high gas flux for carbon dioxide, especially a high permeability for carbon dioxide.

4. Process according to claim 3, characterized in that the membrane has a high gas flux for dimethyl ether, especially a high permeability for dimethyl ether.

5. Process according to claim 1, characterized in that the membrane comprises at least a polymer, especially polydimethylsiloxane.

6. Process according to claim 1, characterized in that the separation step (23) produces a predominantly hydrogen- and carbon monoxide-containing retentate (16) and a predominantly dimethyl ether- and carbon dioxide-containing permeate (15).

7. Process according to claim 6, characterized in that the predominantly hydrogen- and carbon monoxide-containing retentate (16) is fed into the synthesis step (22).

8. Process according to claim 6, characterized in that carbon dioxide (18) is separated from the predominantly dimethyl ether- and carbon dioxide-containing permeate (15).

9. Process according to claim 8, characterized in that carbon dioxide (18) separated from the permeate (15) is used for production (21) of synthesis gas.

10. Process according to claim 1, characterized in that the liquid and gaseous phases are separated before the separation step (23).

11. Process according to claim 1, characterized in that dimethyl ether can be converted into a product containing olefins, particularly ethylene and/or propylene, wherein dimethyl ether is fed to the synthesis of olefins directly or only carbon dioxide is separated from the permeate, before the permeate is fed to the synthesis of olefins.
